(19) United States
(12) Patent Application Publication
(10) Pub. No.: US 2023/0090858 A1
(43) Pub. Date: Mar. 23, 2023

(54) ANALYSIS OF PLEURAL LINES FOR THE DIAGNOSIS OF LUNG CONDITIONS

(71) Applicant: The Trustees of the University of Pennsylvania, Philadelphia, PA (US)

(72) Inventors: Chandra M. Sehgal, Wayne, PA (US); Laith Riyadh Sultan, Exton, PA (US); Theodore William Cary, Philadelphia, PA (US)

(21) Appl. No.: 17/951,985

(22) Filed: Sep. 23, 2022

Related U.S. Application Data
(60) Provisional application No. 63/247,362, filed on Sep. 23, 2021.

(51) Int. Cl. G06T 7/00; G06T 7/11; G16H 50/20; G06T 7/62

(52) U.S. Cl. CPC G06T 7/0012; G06T 7/11; G16H 50/20; G06T 7/62; G06T 2207/20081; G06T 2207/30061; G06T 2207/10132

(57) ABSTRACT

Methods and systems are described for determining a condition of a lung. An example method may comprise receiving imaging data indicative of a lung of a subject, determining at least one pleural line region in the imaging data, determining one or more values of one or more morphological features of the at least one pleural line region, and sending, based on the one or more values of one or more morphological features, an indication of a condition of the lung.

### CROSS REFERENCE TO RELATED APPLICATIONS

This application claims the benefit of U.S. Provisional Application No. 63/247,362 filed Sep. 23, 2021, which is incorporated herein by reference in its entirety for any and all purposes.

### BACKGROUND

Pleural thickening is usually accompanied by tissue scarring often caused by acute inflammation of the pleura. In the normal lung, the pleural line appears as a thin curvilinear opaque lining about 1 to 2 mm in thickness, completely continuous and well defined. COVID-19 is a pleural-based disease, and when patients are infected by it, their pleura are thickened and inflamed. The pleura becomes gradually disrupted as the inflammatory process progresses. The pleural line changes, therefore, carry diagnostic information that are highly suggestive of COVID-19, yet not actively monitored. Thus, there is a need for a more sophisticated analysis of pleural lines for diagnosing lung conditions.

### SUMMARY

Methods and systems are described for determining a condition of a lung. An example method may comprise receiving imaging data indicative of a lung of a subject, determining at least one pleural line region in the imaging data, determining (e.g., computing, calculating) one or more values of one or more morphological features of the at least one pleural line region, and sending, based on the one or more values of one or more morphological features, an indication of a condition of the lung.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to limitations that solve any or all disadvantages noted in any part of this disclosure.

Additional advantages will be set forth in part in the description, which follows or may be learned by practice. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate embodiments and, together with the description, serve to explain the principles of the methods and systems.

FIG. 1A shows a confirmed COVID-19 case with pleural thickening and irregularity detected by semiautomated segmentation.

FIG. 1B shows an example of a normal case where the pleural line is outlined by semiautomated segmentation.

FIG. 2 shows a comparison of computer-based pleural line (p-line) features of COVID-19 and normal cases.

FIG. 3 shows a comparison of computer-based echo-line features for COVID-19 and normal cases.

FIG. 4A illustrates a COVID-19 case diagnosed correctly by quantitative echo-line analysis.

FIG. 4B illustrates a normal case diagnosed correctly by quantitative echo-line analysis.

FIG. 5 shows a comparison of the ROC curves for equally weighted p-line features and echo-line features showing the outperformance of p-line features in differentiating COVID-19 from normal.

FIG. 6 shows a box and whisker graph comparing the separation power of the 2 feature groups.

FIG. 7A shows an example of a COVID-19 confirmed case showing pleural thickening and irregularity with the presence of focal B-lines.

FIG. 7B shows an example of a normal lung image showing a thin and well-defined pleural line and A-lines.

FIG. 8 is a block diagram illustrating an example computing device.

### DETAILED DESCRIPTION OF ILLUSTRATIVE EMBODIMENTS

Significant pathological changes associated with COVID-19 and/or other conditions can be determined by quantitative analysis of pattern changes in the pleural line characteristics of lung ultrasound. The quantitative analysis of a pleural line involves at least two main steps: image segmentation and extraction of features that describe the pleural line changes related to a specific condition, such as COVID-19.

Pleural lines (e.g., or pleural line regions) may be segmented. If the segmentation is semi-automated, the segmentation may be based on selection using a “wand” tool. Following the selection of a pixel within the pleural line with the wand (e.g., or cursor), the algorithm automatically grows the region to include object pixels of similar grayscale within a tolerance range. Only minimal input from the user is requested: to click and then validate the segmentation, making corrections in a few cases where the segmented margin is not acceptable.

Following the detection of the pleural line (p-line), a computing device may extract a variety of features, such as morphological features (e.g., features related to shape), quantitative features describing thickness, margin morphology, brightness, heterogeneity, and/or the like. The features and their formulas are described in Table 3. The thickness parameters may measure the nonuniform widening of the pleural line. The margin morphology features, including tortuosity and nonlinearity, may measure irregularities of the pleural line margin shape. The last group includes grayscale features derived from the first-order histogram to calculate the mean brightness and heterogeneity.

For echo-lines analysis, regions of interest (ROIs) defining lung areas showing A-lines in normal cases and B-lines in COVID-19 cases may be manually outlined by an expert user. Both types of lines were collectively called echo-lines. Quantitative features were extracted as grayscale first-order statistics, and determined by runlength and gray-level co-occurrence matrices (GLCM) (16). The seven image features measured are listed in Table 1 and described in Table 4.

The disclosure may quickly segment and measure features of pleural lines for automatically diagnosing pleural disease, like COVID-19, reducing user variability and improving the diagnostic accuracy of lung ultrasound.

The disclosed techniques improve upon conventional techniques. Some of the specific differences are as follows.

B-lines vs. p-lines: Except for qualitative assessment of pleural line thickening, much of the current diagnosis is based on the interpretation of B-lines in lung ultrasound images. The disclosed approach is primarily based on analyzing pleural lines, and the preliminary results show that its diagnostic performance is better than that based on B-lines.

Quantitative vs. qualitative: The disclosed approach is quantitative as opposed to qualitative interpretation of the images used clinically.

The disclosed approach may use semi-automated detection of pleural lines as opposed to visual inspection of the images.

Feature engineering: The disclosed approach involves identifying specific ultrasound image features that differentiate normal and abnormal pleural lines.

The disclosed techniques are explained in greater detail and may include at least the following the following aspects.

1. A semi-automated method of connecting pixels of the same properties to detect pleural lines (e.g., or pleural line region). The properties may be defined by gray level values and their distribution pattern (texture).

2. The detected object pixels that defined the pleural lines include enough information to engineer features for learning a predictive model of COVID-19 and other inflammatory conditions.

3. The engineered features (a) to (g) may be used to quantify the morphology and physical characteristics of pleural lines on the image and may include at least the following:

a. Thickness—morphology feature, physical measurement

b. Thickness variation—morphology feature

c. Margin tortuosity—morphology feature, shape complexity

d. Nonlinearity—morphology feature, shape complexity

e. Projected intensity deviation—morphology feature, transverse echo variation

f. Brightness—grayscale feature

g. Brightness deviation (Heterogeneity)—grayscale feature

4. These features can be used individually or as an ensemble to measure and assess inflammatory changes in the pleural line caused by COVID-19, pneumonia, and other conditions.

5. The features may be weighted (e.g., equally weighted) and/or used for training in machine learning, to build predictive models to optimize the diagnosis of COVID-19, other forms of pneumonia, and/or other conditions.

6. The engineered features are highly predictive on their own. The appropriate machine learning methods may depend on the number of cases available for training, but in early studies using supervised learning on the engineered features, diagnosis is nearly perfect, proving that the pleural lines contain sufficient information to diagnose the disease. With more cases, more automation is possible so the method could transition from semi-automated to automated.

7. Pleural lines may be combined with B-line analysis to enhance diagnosis.

Details of a study related to the disclosed techniques are described as follows. The disclosed techniques may include any combination of any of the features and/or techniques described below.

Background and objective: Lung ultrasound is an inherently user-dependent modality that could benefit from quantitative image analysis. In this pilot study, we evaluate the use of computer-based pleural line (p-line) ultrasound features in comparison to echo-line features to test the hypothesis that p-line thickening and irregularity are highly suggestive of coronavirus disease 2019 (COVID-19) and can be used to improve the disease diagnosis on lung ultrasound.

Methods: Twenty lung ultrasound images, including normal and COVID-19 cases, were used for quantitative analysis. p-lines were detected by a semiautomated segmentation method. Seven quantitative features describing thickness, margin morphology, and echo intensity were extracted. Echo-lines were outlined, and texture features based on run-length and gray-level co-occurrence matrix were extracted. The diagnostic performance of the 2 feature sets was measured and compared using receiver operating characteristics curve analysis. Observer agreements were evaluated by measuring interclass correlation coefficients (ICC) for each feature.

Results: Six of 7 p-line features showed a significant difference between normal and COVID-19 cases. Thickness of p-lines was larger in COVID-19 cases (6.27±1.45 mm) compared to normal (1.00±0.19 mm), P<0.001. Among features describing p-line margin morphology, projected intensity deviation showed the largest difference between COVID-19 cases (4.08±0.32) and normal (0.43±0.06), P<0.001. From the echo-line features, only 2 features, gray-level non-uniformity and run-length non-uniformity, showed a significant difference between normal cases (0.32±0.06, 0.59±0.06) and COVID-19 (0.22±0.02, 0.39±0.05), P=0.04, respectively. All features together for p-line showed perfect sensitivity and specificity of 100%; whereas, echo-line features had a sensitivity of 90% and specificity of 70%. Observer agreement for p-lines (ICC=0.65 to 0.85) was higher than for echo-line features (ICC=0.42 to 0.72).

Conclusion: P-line features characterize COVID-19 changes with high accuracy and outperform echo-line features. Quantitative p-line features are promising diagnostic tools in the interpretation of lung ultrasound images in the context of COVID-19. Additional details are provided as follows.

Coronavirus disease 2019 (COVID-19) was declared a pandemic caused by severe acute respiratory syndrome coronavirus 2 (SARS-CoV-2) after spreading to >180 countries by March 2020,¹ with >100 million cases confirmed and 2 million deaths.² Medical imaging plays a major role in COVID-19 diagnosis and management.³ The most common modality for imaging COVID-19 patients is chest x-ray, which detects presence of a local or bilateral patchy shadowing infiltrates, but it is known for low sensitivity, with findings absent in >40% of the cases.⁴ Computed tomography (CT) scan has higher sensitivity and shows ground-glass opacities⁵ and, therefore, has been used in different therapeutic and triage strategies since the outbreak started.⁶ The use of chest CT remains limited because of radiation exposure concerns and lack of availability in overextended healthcare facilities.⁷ In the critically ill, the transport of unstable patients and exposure of infected patients also may outweigh the clinical benefit. There continues to be a need for alternative imaging methods that enable quick, low-cost, easy-to-use evaluation of COVID-19 patients.

Lung ultrasound is a promising tool for lung monitoring in the intensive care unit (ICU),^{8,9} particularly for assessing lung aeration.¹⁰⁻¹² A decade of clinical and physical studies demonstrates that lung ultrasound can detect interstitial lung disease, subpleural consolidations, and acute respiratory distress syndrome with different etiologies. With new COVID-19, evolving evidence from clinical practice and several studies shows the usefulness of lung ultrasound for the management of pneumonia, from diagnosis to monitoring and follow-up.¹³⁻¹⁷ Characteristic ultrasound findings have been reported that can help physicians detect and stage the disease, tracking its progression.¹³ The common patterns observed include irregular, thickened pleural lines, multiple B-lines (vertical artifacts) ranging from focal to diffuse, which reflect the stage of inflammatory lung disease. These vertical artifacts of different shapes and lengths occur when the lung loses normal aeration but is not completely consolidated. These findings are usually bilateral with posterior basal predominance in the lungs.

Although there is a general consensus on lung ultrasound's usefulness for COVID-19 pneumonia,¹⁸ it is an inherently user-dependent modality and, without proper training, could result in errors.¹⁹ Limited experience with COVID-19 further adds to the challenge of using lung ultrasound effectively. To overcome these limitations, a number of studies have evaluated quantitative methods for assessing lung ultrasound.²⁰⁻²² These studies assess the use of automated detection of the B-line to provide critical visual information to clinicians in real-time for diagnosis.

In this disclosure, a comprehensive approach is proposed for detecting sonographic features, defining characteristics of a pleural line (p-line) and echo-line from B-lines quantitatively. Thickening of the p-line with irregular margin is highly suggestive of COVID-19.^{14-16, 23} Furthermore, the presence of B-lines influences the p-line characteristics, as these patterns originate from the p-line itself. A-lines represent a more reflective p-line, correlating with the brighter p-line observed normally. The aim of the disclosed techniques is to demonstrate the proof of concept of using quantitative analysis of p-lines for diagnosis and monitoring of COVID-19 by ultrasound imaging. The changes in p-line features are compared to the echo-lines features extracted from A- and B-lines for differentiating COVID-19 cases from normal individuals. The diagnostic performance and the observer variability of these features used individually or as a group was assessed for COVID-19 diagnosis.

A retrospective pilot study was conducted on 20 B-mode ultrasound images that were used to evaluate the proposed quantitative analysis. Ten images were acquired from COVID-19 patients and another independent 10 images were acquired from normal cases Qualitative imaging findings and the results of diagnostic reverse transcription polymerase chain reaction tests were included with the images. The images were received for analysis, and analyzed without patient-related information. The images usually are acquired in a video clip format that includes many frames of the scanned area. Before quantitative analysis, each observer identifies and selects an image from the clip to choose the highest-quality image for making quantitative measurements.

The quantitative analysis involving image segmentation and feature extraction was performed using software written in IDL (Interactive Data Language; version 8.5, L3Harris Geospatial, Boulder, Colo., USA).

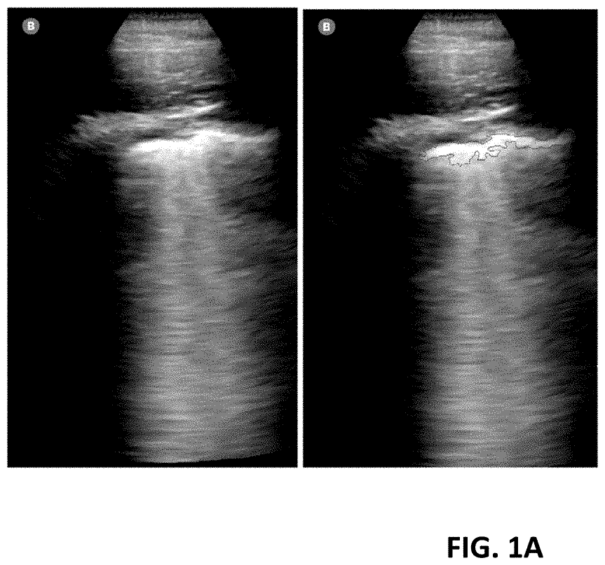

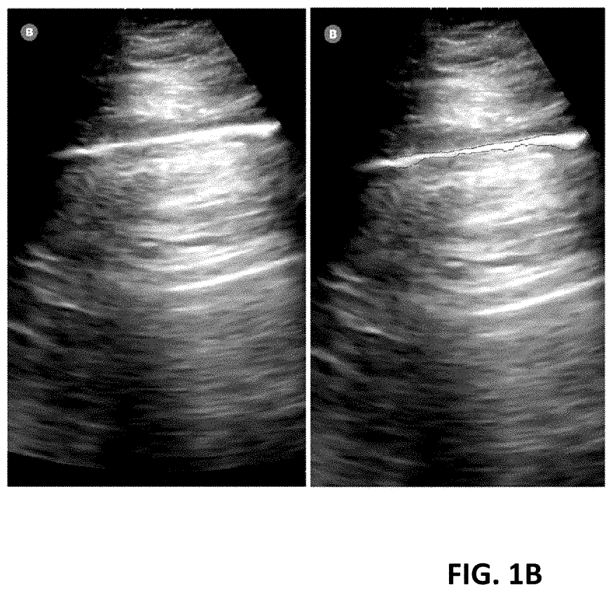

The p-lines were segmented by a “wand” semiautomatic segmentation tool developed by the authors. This semiautomated segmentation approach is the simplest form of “region growing.”²⁴ Following selection of a pixel seed by a user with a single mouse click, the algorithm automatically grows the region to include object pixels of similar grayscale within a tolerance range, ±10 gray levels by default. Only minimal input from the user may be needed: to click and then validate the segmentation, making corrections in a few cases where the segmented margin was not acceptable. FIGS. 1A-B show examples of p-line segmentation in images of COVID-19 and normal cases.

Following the detection of the p-line, the software extracts quantitative features describing the depth (thickness), margin morphology, brightness, and heterogeneity. The features and their formulas are described in Table 3. In summary, the thickness parameters measure the nonuniform widening of the lung pleura represented by the p-line. The margin morphology features, including tortuosity, projected intensity deviation (PID), and non-linearity, measure irregularities of the p-line margin shape. The last group of grayscale features is derived from the first-order histogram to calculate the mean brightness and heterogeneity.

In this study we introduce a predictive model, built on quantitative echo-line features, that serves as an alternative to visual clinical assessment where an observer looks for A-lines in normal lungs and B-lines in COVID-19 cases. Regions of interest (ROIs) defining lung areas showing A-lines in normal cases and B-lines in COVID-19 cases were outlined manually by an expert user. Quantitative features were extracted from the lung images ROIs as grayscale first-order statistics, and determined by run-length and gray-level co-occurrence matrices (GLCM).²⁵ The 7 image features measured are described in Table 4. In brief, the echo-line features computation represents the real texture of the tissue by quantifying non-deterministic properties that govern the distributions and relationships between the grey levels of the ultrasound image. The quantitative features measured demonstrate the changes in lung tissue texture, and should capture and quantify the prominence of A- and B-lines used by humans to distinguish lung ultrasound patterns found in COVID-19 patients from the appearance of normal lung.

Standard descriptive statistics were computed for the features extracted from the images: arithmetic mean and standard error. The 2-tailed Student's t test of unequal variance was used to determine the statistical significance of the difference between 2 groups. P<0.05 was considered significant.

The diagnostic performance for individual ultrasound features includes area under the curve (AUC) and sensitivity and specificity at the Youden Index. To assess the overall diagnostic performance of features as groups, features were normalized relative to their maximum and minimum values and assigned equal weights to calculate the likelihood of COVID-19. The averages of p-line and echo-line features after normalization were used independently to test the accuracy of each group for differentiating normal from COVID-19 cases by receiver operating characteristics analysis.

To evaluate the reproducibility of the analysis, observer agreement for the same individual (intraobserver) and between 2 individuals (interobserver) were measured. Two physicians with >10 years of experience in ultrasound imaging analyzed the same image set. The observers were blinded to the diagnosis and any other patient-related information. Both observers performed the quantitative analysis, which includes selection of the seed for p-line semiautomatic segmentation and outlining the ROI for the echo-line analysis. No visual interpretation was made by the observers. To account for the variation related to selection of images, the analysis was repeated where one observer selected the images for analysis from a video clip. Intra- and interobserver observer agreement in feature analysis was measured by interclass correlation coefficient.^{26,27}

FIGS. 1A-B show examples of lung ultrasound images demonstrating the semiautomated detection method of pleural lines. FIG. 1A shows a confirmed COVID-19 case with pleural thickening and irregularity. The right panel of FIG. 1A shows the pleural line detected with the semiautomated segmentation. FIG. 1B shows an example of a normal case where a pleural line is outlined by semiautomated segmentation. The segmented pleural line is shown in the right panel of FIG. 1B.

Qualitative image findings were provided by an emergency physician experienced with lung ultrasound for both normal and COVID-19 images. Normal images were described as having a thin, well-defined p-line with the presence of A-lines, whereas findings related to COVID-19 patients included thick and irregular p-lines with the presence of B-lines. COVID-19 images were from patients 8 to 12 days following COVID-19 diagnosis. COVID-19 patients showed mild to moderate symptoms. All had bilateral pneumonia with good oxygen saturation. Patient ages ranged from 30 to 60 years.

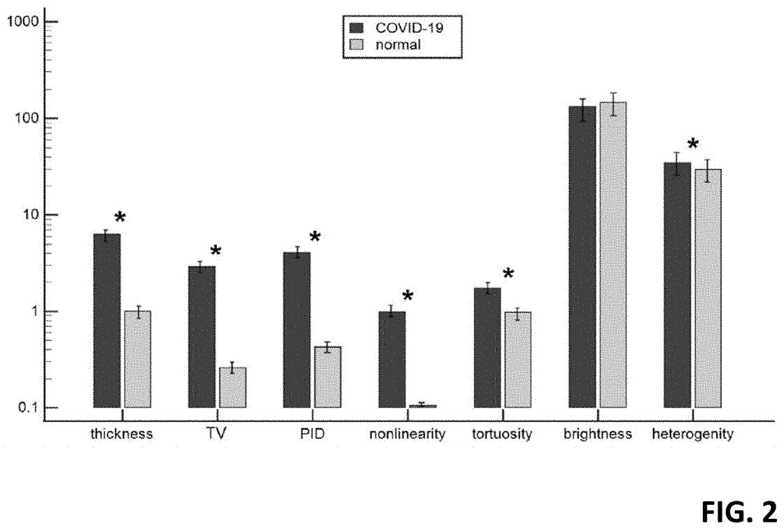

From the 7 p-line features extracted from lung ultrasound images, 6 showed statistically significant differences (P<0.05) between normal and COVID-19 cases. FIG. 2 shows a comparison of the mean and standard errors for each feature of COVID-19 cases to those of normal cases. The thickness of p-lines was larger on average in COVID-19 cases (6.27±1.45 mm) compared to normal (1.00±0.19 mm), P<0.001. P-line thickness variation was also larger on average in COVID-19 cases, 2.86±0.64 mm compared to 0.26±0.07 mm, P<0.001.

Among features describing margin morphology, PID showed the largest difference between the 2 groups in COVID-19 cases, 4.08±0.32 compared to 0.43±0.06 in normal cases, P<0.001. Nonlinearity was also larger for the COVID-19 cases, 0.98±0.10 compared to 0.01±0.01, P<0.001. Margin tortuosity was about 2 times larger in COVID-19 cases, 1.73±0.09 compared to 0.97±0.01, P<0.001.

The mean p-line brightness was higher in normal cases (145.98±12.15) compared to COVID-19 cases (132.61±7.51) but was not significant (P=0.14). On the other hand, p-line heterogeneity increased significantly in COVID-19 cases (34.67±2.07) compared to normal cases (29.41±2.87), P=0.04.

The sensitivity, specificity, and AUC for each p-line feature are summarized in Table 1. Notably, p-line margin morphology features showed the highest performances, with specificities ranging from 90 to 100% and sensitivities of 100%. Similarly, thickness features showed high sensitivity values ranging from 80 to 90% and a specificity value of 100%. On the contrary, p-line brightness and heterogeneity had lower performance than other features, with sensitivity of 60% (Table 1).

FIG. 2 shows a comparison of computer-based pleural line (p-line) features of COVID-19 and normal cases. Bars represent mean±SE of each feature. Red bars represent COVID-19 cases. Light blue bars represent normal cases. The ‘*’ indicates a statistically significant difference (P<0.05). Notably, 6 of 7 p-line features showed a statistically significant difference between the 2 groups. The results are from coronavirus disease in 2019. The statistically significant features included the thickness, thickness variation (TV), projected intensity deviation (PID), nonlinearity, tortuosity, and heterogeneity. Any one or combination of these features may be used to identify a condition of a subject.

Table 1 shows the diagnostic performance for quantitative pleural line (p-line) features and echo-line features showing AUC, sensitivity, and specificity for each feature. (AUC, area under the curve; GLCM, gray-level co-occurrence matrix mean; GLNU, gray-level non-uniformity; RLNU, run-length non-uniformity.)

TABLE 1

| | AUC | Sensitivity % | Specificity % |
|---|---|---|---|
| P-line features | | | |
| Thickness (mm) | 0.91 | 80 | 100 |
| Thickness variation (mm) | 0.98 | 90 | 100 |
| Margin tortuosity | 1 | 100 | 100 |
| Projected intensity deviation | 1 | 100 | 100 |
| Nonlinearity | 0.97 | 100 | 90 |
| Brightness | 0.66 | 60 | 90 |
| Heterogeneity | 0.71 | 100 | 50 |
| Echo-line features | | | |
| Echo intensity | 0.52 | 60 | 10 |
| Tissue heterogeneity | 0.64 | 80 | 50 |
| GLNU | 0.74 | 80 | 70 |
| RLNU | 0.75 | 90 | 70 |
| GLCM | 0.71 | 80 | 60 |
| Homogeneity | 0.69 | 40 | 100 |
| Entropy | 0.68 | 60 | 80 |

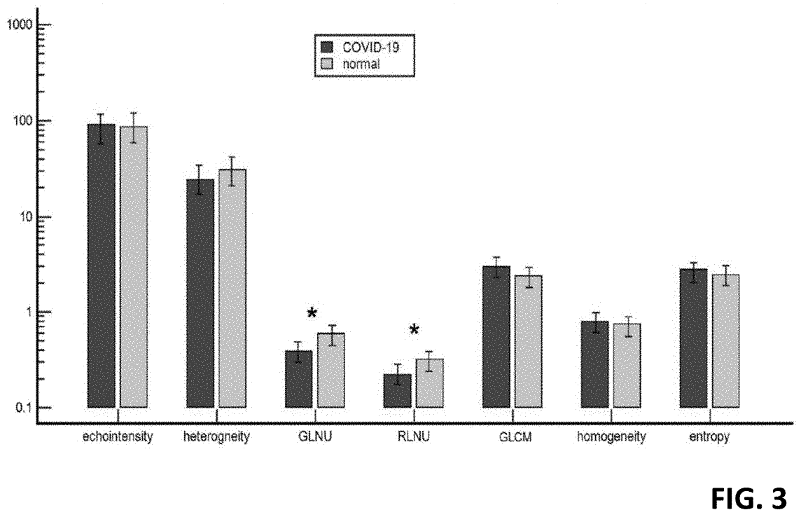

Only 2 of the 7 features of echo-line texture measurements showed a significant difference between COVID-19 and normal cases (e.g., as shown in FIG. 3). Run-length matrix features including gray-level non-uniformity (0.32±0.06) and run-length non-uniformity (0.59±0.06) were both significantly higher in normal cases compared to those diagnosed with COVID-19 (0.22±0.02, 0.39±0.05, P=0.04), respectively.

Echo-line heterogeneity was also higher in normal cases (30.78±3.91) compared to the heterogeneity observed in COVID-19 cases (24±0.07), P=0.08. Conversely, mean echo intensity level was higher in the COVID-19 echo-line (90.96±13.06) compared to normal cases (85.40±8.78); however, this difference did not reach a significance level, with P=0.73.

Gray-level co-occurrence matrix (GLCM) features were all higher for COVID-19 cases than for normal cases; however, none reached significance. GLCM mean in COVID-19 patients was 2.98±0.29 compared to 2.37±0.23, P=0.09. The other 2 GLCM features, entropy and homogeneity, were both higher in COVID-19, 2.98±0.29 and 2.78±0.13, compared to normal cases, 2.37±0.23 and 2.45±0.18, P=0.08 and P=0.12, respectively.

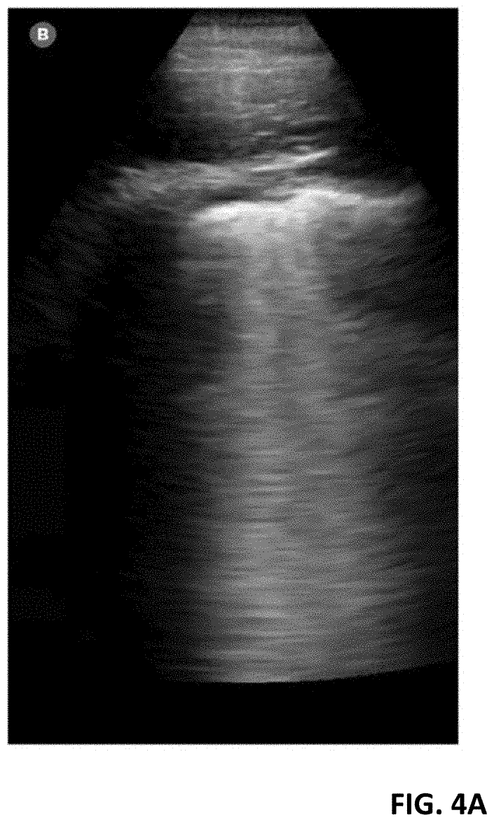

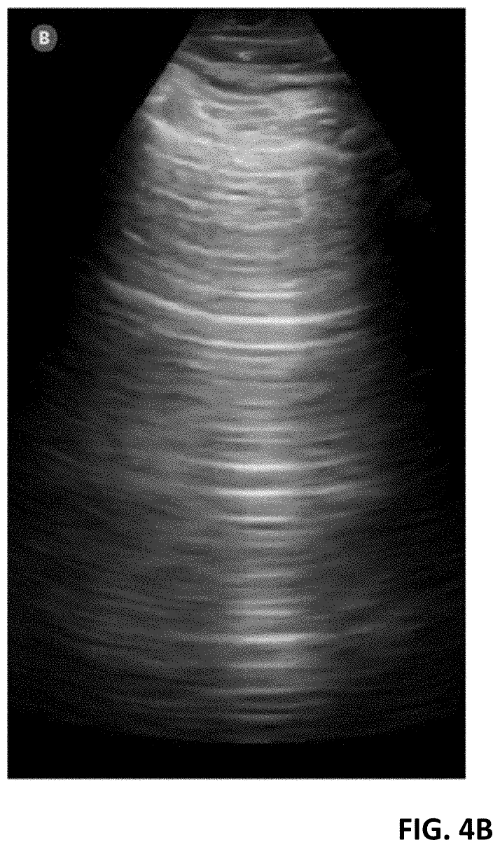

Table 1 summarizes the sensitivity, specificity, and AUC for each echo-line texture feature. Run-length texture features showed the highest performance with sensitivity ranging between 80 and 90% and specificity of 70%. Echo-line heterogeneity had a sensitivity of 80% and specificity of 60%. Of the GLCM features, GLCM mean had the highest sensitivity (sensitivity=80%) and GLCM homogeneity was the most specific feature (specificity=100%). Echo intensity had the lowest performance among other features with a sensitivity of 60% and specificity of 10%. FIGS. 4A-B show an example of 2 cases that were diagnosed correctly by Echo-line analysis. The quantitative analysis showed that the case diagnosed with COVID-19 has higher echogenicity and lower heterogeneity compared to the opposite pattern observed in the normal case.

FIG. 3 shows a comparison of computer-based echo-line features for COVID-19 normal cases. Bars represent the mean ±SE of each feature studied. Red bars represent COVID-19 cases. Light blue bars represent normal cases. The ‘*’ indicates a statistically difference (P<0.05). (COVID-19, coronavirus disease 2019; GLCM, gray-level co-occurrence matrix mean; GLNU, gray-level non-uniformity; RLNU, run-length non-uniformity; echo-line).

FIGS. 4A-B show examples of cases that were diagnosed correctly by quantitative echo-line analysis. The 2 cases showed different texture patterns. FIG. 4A illustrates a COVID-19 case with higher echogenicity and lower heterogeneity, RLNU, and GLNU. FIG. 4B illustrates a normal case with lower echogenicity and higher heterogeneity, RLNU, and GLNU. (COVID-19, coronavirus disease 2019; GLNU, gray-level non-uniformity; RLNU, run length non-uniformity; echo-line).

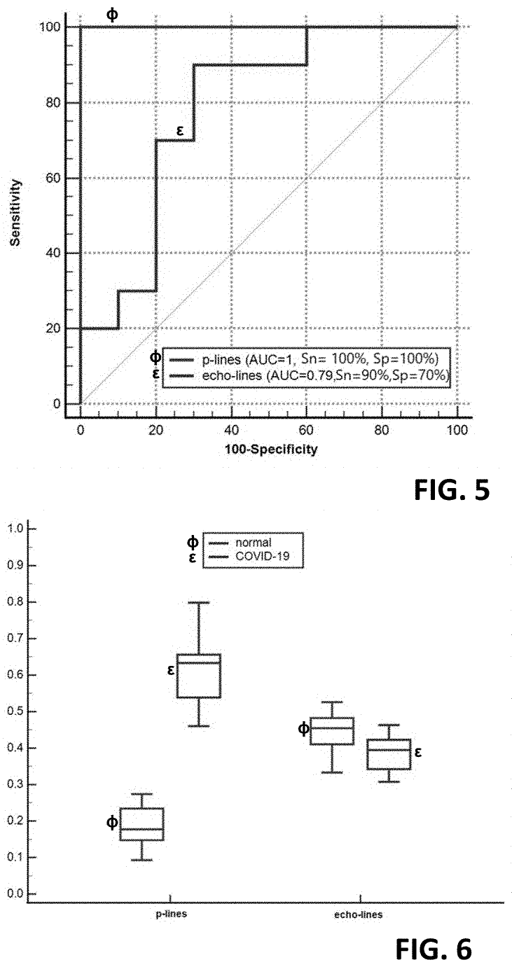

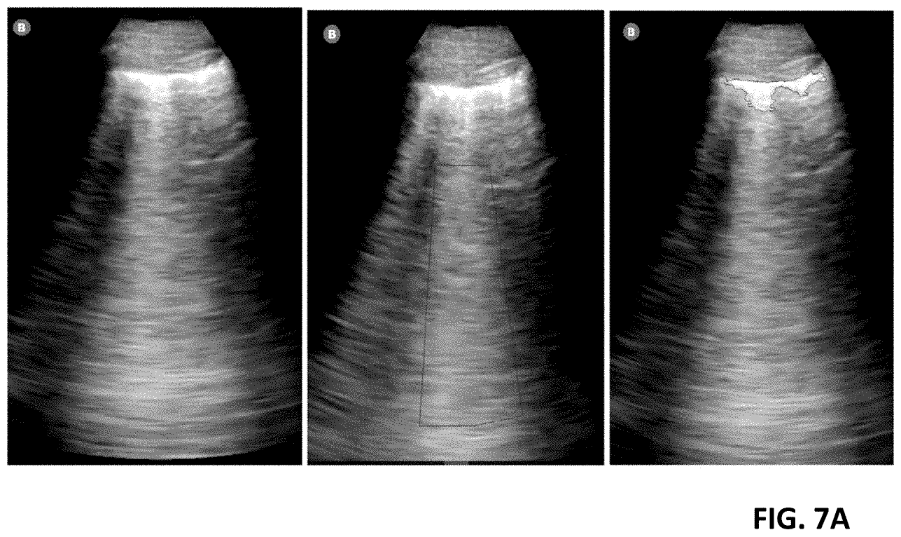

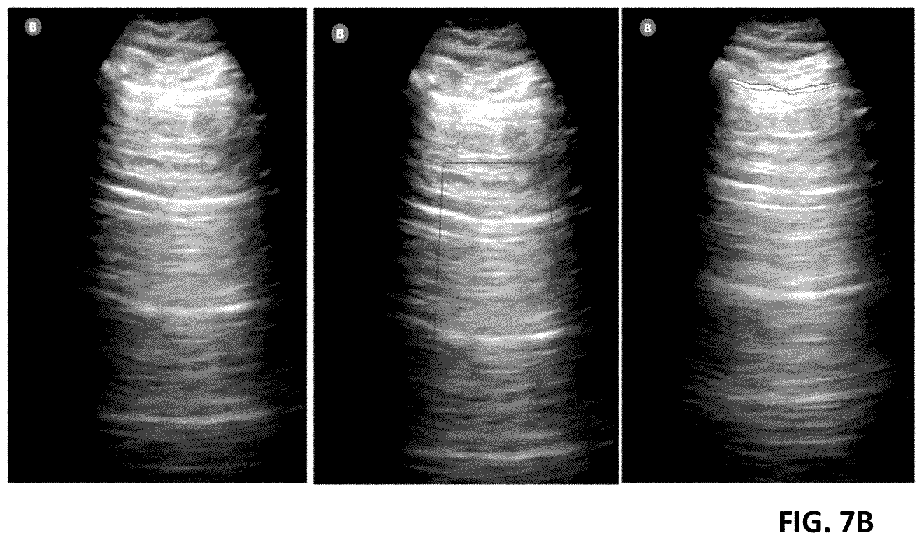

Equally weighted p-lines features when used together were better at differentiating normal cases from COVID-19, achieving a perfect performance with AUC=1.0 compared to AUC=0.79 with equally weighted echo-line features (e.g., as shown in FIG. 5). p-line features had perfect sensitivity of 100% and specificity of 100% in separating the 2 groups, whereas echo-line features had a sensitivity of 90% and specificity of 70%. The p-line model showed a superior separation of the 2 groups compared to the model, whose features were closely distributed and even overlapping (e.g., as shown in FIG. 6). FIGS. 7A-B show the cases that were incorrectly identified using echo-line features, whereas p-line features identified the cases correctly. FIG. 7A is an example of a COVID-19 case where the model built on quantitative p-line features correctly diagnosed, but the model from echo-line features was incorrect, diagnosing the case as normal. Similarly, in FIG. 7B, a normal case was labeled incorrectly as COVID-19, but p-line features extracted by the computer correctly diagnosed the case. The misdiagnosed cases showed an echo-line pattern overlapping between the two groups, COVID-19 and normal cases.

FIG. 5 shows a comparison of the ROC curves for equally weighted p-line features and echo-line features showing outperformance of p-line features in differentiating COVID-19 from normal. (AUC, area under the curve; COVID-19, coronavirus disease 2019; p-lines, pleural lines; ROC, receiver operating characteristics; sn, sensitivity; sp, specificity).

FIG. 6 shows a box and whisker graph comparing the separation power of the 2 feature groups. p-line features show a clear separation between normal and COVID-9 cases whereas cases are closely distributed with echo-line features. X-axis represents equally weighted normalized features. (COVID-19, coronavirus disease 2019; p-line, pleural line).

The individual observer analyzed the same images 2 weeks following the first analysis. Interclass correlation coeffecients (ICCs) between the 2 analyses on average for p-line features showed excellent agreement of ICC of 0.85±0.09 (Table 2). ICC for individual p-line features ranged from ICC of 0.72 (good agreement) for heterogeneity, to ICC of 0.95, excellent agreement, for non-linearity.

On the other hand, echo-line features showed good agreement on average with ICC of 0.71±0.15. ICC ranged from 0.52, fair agreement with run-length non-uniformity, to excellent agreement (ICC=0.92) for echo intensity (Table 2).

High agreement levels were recorded between 2 observers who analyzed the same set of images (Table 2). The average ICC for p-line features was 0.83±0.12, excellent agreement. The feature that showed the highest agreement was p-line thickness with ICC of 0.98, whereas p-line brightness showed the lowest agreement (ICC=0.63).

For echo-line, overall agreement was good with ICC=0.67±0.10. The highest agreement in a feature was in GL non-uniformity (ICC=0.87); whereas, the lowest was seen in echo-line heterogeneity with ICC=0.56 (Table 2).

Intra- and interobserver agreements were calculated for analyses using another set of images (Table 2). For both intra- and interobserver, ICCs for p-line features on average were good (ICC of 0.65-0.71). On the other hand, the agreement for echo-line features was lower, with ICC of 0.45 to 0.59, fair agreement.

Ultrasound is an ideal imaging tool for COVID-19 diagnosis because of its high sensitivity, safety, portability, and affordability.²⁸ However, a significant disadvantage is that it is highly user-dependent, and not all clinicians have training in performing lung ultrasound and reading the images. Operator experience may also affect specificity because an expert will correlate different lung ultrasound patterns with different disease processes.²⁹ To overcome these problems, a number of studies have evaluated quantitative assessment methods²⁰⁻²² to help physicians in image interpretation. However, the focus of these studies is limited to developing semi-quantitative scoring systems based on B-line identification. There remains a need for a comprehensive approach that includes quantitative analysis of both the p-line and echo-line indicative of COVID-19.

FIG. 7A shows an example of a COVID-19 confirmed case showing pleural thickening and irregularity with the presence of focal B-lines. Quantitative pleural line features detected the case accurately as COVID-19, whereas echo-line features incorrectly identified the case as normal. FIG. 7B shows a normal lung image showing a thin and well-defined pleural line and A-lines. Quantitative pleural line features detected the case accurately as normal, whereas echo-line features incorrectly identified the case as COVID-19. (COVID-19, coronavirus disease 2019; echo-line).

COVID-19 is a pleural-based disease, and when patients are infected by it, their pleura is thickened and inflamed. Pleural thickening is usually accompanied by tissue scarring,^{30,31} often caused by acute inflammation of the pleura. In the normal lung, the p-line appears as a thin curvilinear opaque lining 1 to 2 mm in thickness, completely continuous and well-defined. It becomes gradually disrupted as the pathological condition worsens. This study focused on evaluating these pathological changes using ultrasound, which is ideally suited for imaging small structures. The idea central to the study is that significant pathological changes associated with COVID-19 can be determined by quantitative analysis of pattern changes in the p-line characteristics of lung ultrasound. A comparison between COVID-19 and non-COVID-19 cases showed that it is possible to detect and characterize p-line changes related to COVID-19 with high accuracy using computer-derived image features. The semiautomated segmentation tool detected the p-line margins with fine details capturing the changes in margin morphology. Margin shape features including margin tortuosity, projected intensity deviation, and non-linearity were significantly higher in COVID-19 cases, correlating closely with the irregularity and changes in p-line shape related to the inflammatory process reported in previous studies. Additionally, the mean thickness of p-lines was measured as >6 times greater than p-line thickness in normal cases, which is also consistent with qualitative clinical assessments of COVID-19 cases on lung ultrasound. Notably, the p-lines showed less mean brightness and higher heterogeneity in COVID-19 compared to normal. These observed changes are consistent with the presence of inflammatory cells and edema in COVID-19 cases, which cause the p-line to lose its high intensity and uniform appearance seen in normal lungs.

Table 2 shows a summary of the observer agreements in p-line ultrasound features and echo-line features using the same and different images of the subject. (GLCM, gray level co-occurrence matrix mean; GLNU, gray-level non-uniformity; ICC, interclass correlation coefficients; p-line, pleural line; RLNU, run-length non-uniformity.)

TABLE 2

| | Same image | | Different images | |
|---|---|---|---|---|
| | Intra- | Inter- | Intra- | Inter- |
| | observer | observer | observer | observer |
| | ICC | ICC | ICC | ICC |
| P-line features | | | | |
| Thickness | 0.85 | 0.98 | 0.65 | 0.92 |
| Thickness | 0.76 | 0.94 | 0.68 | 0.85 |
| variation | | | | |
| Tortuosity | 0.91 | 0.82 | 0.67 | 0.35 |
| Projected | 0.79 | 0.80 | 0.70 | 0.84 |
| intensity | | | | |
| deviation | | | | |
| Nonlinearity | 0.95 | 0.63 | 0.82 | 0.89 |
| Brightness | 0.94 | 0.73 | 0.73 | 0.90 |
| Heterogeneity | 0.72 | 0.88 | 0.33 | 0.31 |
| Average ICC | 0.85 ± 0.09 | 0.83 ± 0.12 | 0.71 ± 0.15 | 0.72 ± 0.27 |
| (± SE) | | | | |
| Echo-line features | | | | |
| Echo | 0.93 | 0.69 | 0.59 | 0.94 |
| intensity | | | | |
| Tissue | 0.82 | 0.56 | 0.33 | 0.66 |
| heterogeneity | | | | |
| GLNU | 0.52 | 0.87 | 0.31 | 0.44 |
| RLNU | 0.55 | 0.61 | 0.41 | 0.40 |
| GLCM mean | 0.70 | 0.71 | 0.49 | 0.76 |
| Homogeneity | 0.67 | 0.67 | 0.54 | 0.45 |
| Entropy | 0.76 | 0.60 | 0.33 | 0.52 |
| Average ICC | 0.71 ± 0.15 | 0.67 ± 0.10 | 0.45 ± 0.11 | 0.59 ± 0.20 |
| (± SE) | | | | |

The sensitivity and specificity for the individual p-line features as well as the overall performance of all the features together were high, thereby confirming their ability to differentiate normal from COVID-19 cases with excellent accuracy. In particular, p-line margin morphology and its thickness were more specific for COVID-19 in comparison to brightness and heterogeneity. The high specificity of quantitative margin morphology features over other features could make these features better suited for diagnostic models of COVID-19.

The echo-line features, extracted from A-lines and B-lines, were also able to detect COVID-19-related changes. However, these features, with AUC approaching 0.79, unlike p-line features, did not have significant discriminatory power to diagnose the cases. The study results show that brighter and more homogenous areas are seen in COVID-19 cases. With progression of the disease, the lungs are filled with inflammatory cells as well as fluids that cause the ultrasound beam to be trapped between the inflammatory cells, producing the vertical artifacts called B-lines.^{12,13} These lines are brighter and more uniform compared to A-lines seen in normal lungs. We were able to quantify these findings but did not observe a significant difference between the 2 groups, and it is unknown if the difference could become significant with more cases. Individual echo-line features exhibited less sensitivity and specificity than p-line features. One reason could be that B-lines are imaging artifacts, and their genesis remains unclear and multifactorial, correlating with various pathological conditions of the lung. In contrast, the p-lines in lung ultrasound represent real physical structures that undergo acute inflammatory changes specific to COVID-19.³² Earlier studies also reported reduced blood flow in the p-line by Doppler images in COVID-19 patients compared to the increased flow seen in other types of viral pneumonia,¹⁴ again because of the acute nature of the disease.

The high observer agreement indicates the consistency and reliability of the quantitative analyses. In all observer variability analyses, p-line features were more stable and consistent compared to the echo-line features, which had a lower agreement between observations. A notable decrease in ICC of >0.15 was observed when different images were selected by the observer for the analysis, suggesting that >15% variation could result from the choice of images used for analysis. The effect of image selection has also been observed with B-lines scoring,³³ where minute differences between images were found to significantly influence the scoring process and, ultimately the final diagnosis. These findings underscore the importance of image selection in clinical settings.

In conclusion, we introduced a computer-based system that captures p-line pattern changes associated with COVID-19 by quantitative analysis of lung ultrasound. Quantitative p-line features showed high accuracy in detecting COVID-19 cases compared to echo-line features, which can be more uncertain. These results suggest that a comprehensive quantitative system that characterizes p-lines would improve the diagnostic accuracy of COVID-19 on lung ultrasound. The automated methods were performed offline but these methods can be easily integrated into the operation of the scanner for real-time bedside assessment. Future studies may incorporate advanced machine learning methods to optimize the p-line detection algorithm for robustness and automation. Given more cases, fully automated machine learning segmentation could be performed. This technology when implemented successfully in clinical practice will increase confidence in diagnosis, especially in low-resource communities around the globe that lack experience in lung ultrasound.

Table 3 provides a description and formulas for pleural-line (p-line) features. Some morphological features may be determined based on fitting a function to measured values associated with a pleural line region. In some scenarios, some of these features may be excluded from the analysis. The morphology features may be determined without using all the features for determining an indication of a condition. For example, thickness may be used to determine thickness variation, but the indication of the condition may not be determined using the thickness but rather the thickness variation. These are example features that may be used for analyzing pleural lines (e.g., or pleural line regions). However, the features and corresponding definitions may vary according to different application and design requirements.

TABLE 3

| Features | Formulas |
|---|---|
| Thickness and thickness variation (mm) For region R = {xᵢ, yᵢ}, ranging over Nₓ horizontal values, the mean thickness is | $\overline{d} = \frac{{\sum }_{x = min ( x )}^{max ( x )} max ( {y}_{x} ) - min ( {y}_{x} )}{{N}_{x}} ,$ |
| the mean of the y ranges taken through | |
| the region at each horizontal coordinate. | ${σ}_{d} = \sqrt{\frac{{\sum }_{x = min ( x )}^{max ( x )} {( max ( {y}_{x} ) - min ( {y}_{x} ) - \overline{d} )}^{2}}{{N}_{x}}}$ |
| Margin tortuosity (unitless) The perimeter of the lesion divided by the circumference of its best-fit ellipse, | $\frac{{P}_{lesion}}{{C}_{ellipse}} ,$ |
| also called elliptically normalized | where P is the region's perimeter and C is the |
| circumference. | circumference of a best-fit ellipse to the region. |
| Projected intensity deviation (PID, gray level) Projected deviation measures the horizontal (transverse) irregularity in a region's gray level by computing the standard deviation of the depth- projected mean intensity. Given image | $\sqrt{\frac{{\sum }_{x = 1}^{{N}_{x}} {( \frac{{\sum }_{y = 1}^{{N}_{y}} {I}_{x , y}}{{N}_{y}} - \frac{{\sum }_{x = 1}^{{N}_{x}} \frac{{\sum }_{y = 1}^{{N}_{y}} {I}_{x , y}}{{N}_{y}}}{{N}_{x}} )}^{2}}{{N}_{x}}}$ |
| image I_{x,y} of dimensions Nₓ, N_{y}, it is | |
| given by the formula to the right. | |
| Non-linearity (unitless) The probability that the points in a | $1 - ( 1 - {e}^{\frac{- {χ}^{2}}{2}} ) = {e}^{\frac{- {χ}^{2}}{2}} ,$ |
| region R = {xᵢ, yᵢ} will lie on their linear | |
| regression fit a + bxᵢ, where the | where χ² is the chi-square of a linear fit, given by: |
| regression minimizes χ². This | |
| probability is the same as the probability of a t test with the 2 degrees of freedom (a, b) on the regression fit. | ${χ}^{2} = arg {min}_{a , b} {{\sum }^{N}}_{i = 1} {( {y}_{i} - a - {bx}_{i} )}^{2}$ |
| Echo intensity mean (μ) and deviation (σ) of pleural line (gray level) First-order histogram features: mean | $μ = \frac{{\sum }_{x}^{{N}_{x}} {\sum }_{y}^{{N}_{y}} {I}_{x , y}}{{N}_{x} {N}_{y}} , σ = \frac{{\sum }_{x}^{{N}_{x}} {\sum }_{y}^{{N}_{y}} \sqrt{{( {I}_{x , y} - μ )}^{2}}}{{N}_{x} {N}_{y}} ,$ |
| brightness of the segmented pleural | |
| lines and the measured variability in | where I_{x,y} is the intensity (grayscale) of the pleural |
| mean brightness, respectively. | line at coordinate [x, y] |

Table 4 provides description and formulas for echo-line features. These are example features that may be used for analyzing echo-lines. However, the features and corresponding definitions may vary according to different application and design requirements.

TABLE 4

| Features | Formulas |
|---|---|
| Echo intensity mean (μ) and deviation (σ) (gray level) First order-histogram features that describe | $μ = \frac{{\sum }_{x}^{{N}_{x}} {\sum }_{y}^{{N}_{y}} {I}_{x , y}}{{N}_{x} {N}_{y}} , σ = \frac{{\sum }_{x}^{{N}_{x}} {\sum }_{y}^{{N}_{y}} \sqrt{{( {I}_{x , y} - μ )}^{2}}}{{N}_{x} {N}_{y}} ,$ |
| the brightness of the tissue (mean) and the | |
| variability in brightness (standard deviation). | where I_{x,y} is the intensity (grayscale) of the |
| | echo line at coordinate [x, y]. |
| Gray-level non-uniformity (unitless) This may be a run-length-matrix (RLM) feature that measures disorderliness of | $\frac{1}{H} {\sum }_{i} {( {\sum }_{j} {p}_{i , j} )}^{2} ,$ |
| homogenous runs of gray along defined | |
| directions. The RLM p gives the length of | where H is the total count of homogenous |
| homogenous runs for each gray level, | runs in p, the run-length matrix defined at left. |
| computed over 8 directions (vertical up and | |
| down, horizontal left and right, diagonals), so | |
| that element (i, j) is the number of | |
| homogeneous runs of j pixels with intensity i. | |
| Run-length non-uniformity (unitless) This may be a run-length matrix (RLM) feature that measures the disorderliness of the | $\frac{1}{H} {\sum }_{j} {( {\sum }_{i} {p}_{i , j} )}^{2} ,$ |
| lengths of homogeneous runs, computed | |
| similarly to gray-level non-uniformity above, | where H is the total count of homogeneous |
| with the inner and outer sums switched. | runs, and p is the RLM defined above. |
| Gray-level co-occurrence matrix mean (unitless) The mean of the GLCM (p), the co- | ${\sum }_{i , j = 0}^{N - 1} {ip}_{i , j} ,$ |
| occurrence matrix which records the counts of | |
| pixel intensity combinations occurring | where p is the GLCM as defined at left. |
| between neighboring pixels, computed over 8 | |
| directions (up and down, left and right, | |
| diagonals). Element (i, j) is the count of times | |
| a pixel of intensity i had a pixel of intensity j | |
| next to it in any direction at some scale. This | |
| may be a measure of orderliness, and records | |
| whether small patterns repeat themselves. | |
| Gray level co-occurrence matrix homogeneity of echo line (unitless) Also called the inverse difference moment, | ${\sum }_{i , j = 0}^{N - 1} \frac{{p}_{i , j}}{1 + {( i - j )}^{2}} ,$ |
| homogeneity increases if the region has less | |
| contrast. | where p is the GLCM as defined above. |
| Entropy (unitless) Larger entropy means the texture is more disordered. | ${\sum }_{i , j = 0}^{N - 1} {p}_{i , j} ( - ln {p}_{i , j} ) ,$ |
| | where p is the GLCM as defined above. |

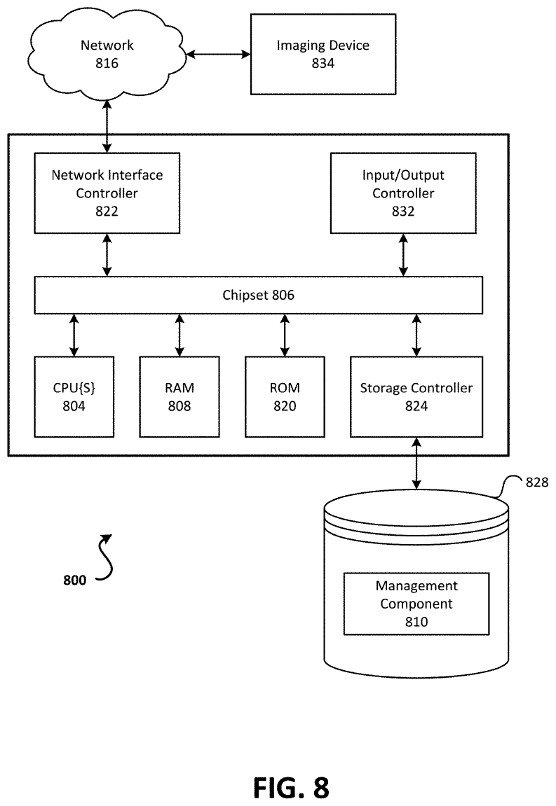

FIG. 8 depicts a computing device that may be used to implement the imaging and/or analysis techniques described herein. The computer architecture shown in FIG. 8 shows a conventional server computer, workstation, desktop computer, laptop, tablet, network appliance, PDA, e-reader, digital cellular phone, or other computing node, that may be utilized to execute any aspect of the computing described herein, such as analyzing images, determining a condition (e.g., indication of a disease), or to output an indication of the condition. Additionally, the computing device may be configured to implement a machine learning model configured to recognize features of pleural lines, categorize images based on imaging features (e.g., pleural lines, morphology of pleural lines), and/or the like.

The computing device 800 may include a baseboard, or “motherboard,” which is a printed circuit board to which a multitude of components or devices may be connected by way of a system bus or other electrical communication paths. One or more central processing units (CPUs) 804 may operate in conjunction with a chipset 806. The CPU(s) 804 may be standard programmable processors that perform arithmetic and logical operations necessary for the operation of the computing device 800.

The CPU(s) 804 may perform the necessary operations by transitioning from one discrete physical state to the next through the manipulation of switching elements that differentiate between and change these states. Switching elements may generally include electronic circuits that maintain one of two binary states, such as flip-flops, and electronic circuits that provide an output state based on the logical combination of the states of one or more other switching elements, such as logic gates. These basic switching elements may be combined to create more complex logic circuits including registers, adders-subtractors, arithmetic logic units, floating-point units, and the like.

The CPU(s) 804 may be augmented with or replaced by other processing units, such as GPU(s) 805. The GPU(s) 805 may comprise processing units specialized for but not necessarily limited to highly parallel computations, such as graphics and other visualization-related processing.

A chipset 806 may provide an interface between the CPU(s) 804 and the remainder of the components and devices on the baseboard. The chipset 806 may provide an interface to a random access memory (RAM) 808 used as the main memory in the computing device 800. The chipset 806 may further provide an interface to a computer-readable storage medium, such as a read-only memory (ROM) 820 or non-volatile RAM (NVRAM) (not shown), for storing basic routines that may help to start up the computing device 800 and to transfer information between the various components and devices. ROM 820 or NVRAM may also store other software components necessary for the operation of the computing device 800 in accordance with the aspects described herein.

The computing device 800 may operate in a networked environment using logical connections to remote computing nodes and computer systems through local area network (LAN) 816. The chipset 806 may include functionality for providing network connectivity through a network interface controller (NIC) 822, such as a gigabit Ethernet adapter. A NIC 822 may be capable of connecting the computing device 800 to other computing nodes over a network 816. It should be appreciated that multiple NICs 822 may be present in the computing device 800, connecting the computing device to other types of networks and remote computer systems.

The computing device 800 may be connected to a mass storage device 828 that provides non-volatile storage for the computer. The mass storage device 828 may store system programs, application programs, other program modules, and data, which have been described in greater detail herein. The mass storage device 828 may be connected to the computing device 800 through a storage controller 824 connected to the chipset 806. The mass storage device 828 may consist of one or more physical storage units. A storage controller 824 may interface with the physical storage units through a serial attached SCSI (SAS) interface, a serial advanced technology attachment (SATA) interface, a fiber channel (FC) interface, or other type of interface for physically connecting and transferring data between computers and physical storage units.

The computing device 800 may store data on a mass storage device 828 by transforming the physical state of the physical storage units to reflect the information being stored. The specific transformation of a physical state may depend on various factors and on different implementations of this description. Examples of such factors may include, but are not limited to, the technology used to implement the physical storage units and whether the mass storage device 828 is characterized as primary or secondary storage and the like.

For example, the computing device 800 may store information to the mass storage device 828 by issuing instructions through a storage controller 824 to alter the magnetic characteristics of a particular location within a magnetic disk drive unit, the reflective or refractive characteristics of a particular location in an optical storage unit, or the electrical characteristics of a particular capacitor, transistor, or other discrete component in a solid-state storage unit. Other transformations of physical media are possible without departing from the scope and spirit of the present description, with the foregoing examples provided only to facilitate this description. The computing device 800 may further read information from the mass storage device 828 by detecting the physical states or characteristics of one or more particular locations within the physical storage units.

In addition to the mass storage device 828 described above, the computing device 800 may have access to other computer-readable storage media to store and retrieve information, such as program modules, data structures, or other data. It should be appreciated by those skilled in the art that computer-readable storage media may be any available media that provides for the storage of non-transitory data and that may be accessed by the computing device 800.

By way of example and not limitation, computer-readable storage media may include volatile and non-volatile, transitory computer-readable storage media and non-transitory computer-readable storage media, and removable and non-removable media implemented in any method or technology. Computer-readable storage media includes, but is not limited to, RAM, ROM, erasable programmable ROM (“EPROM”), electrically erasable programmable ROM (“EEPROM”), flash memory or other solid-state memory technology, compact disc ROM (“CD-ROM”), digital versatile disk (“DVD”), high definition DVD (“HD-DVD”), BLU-RAY, or other optical storage, magnetic cassettes, magnetic tape, magnetic disk storage, other magnetic storage devices, or any other medium that may be used to store the desired information in a non-transitory fashion.

A mass storage device, such as the mass storage device 828 depicted in FIG. 8, may store an operating system utilized to control the operation of the computing device 800. The operating system may comprise a version of the LINUX operating system. The operating system may comprise a version of the WINDOWS SERVER operating system from the MICROSOFT Corporation. The operating system may comprise a version of the UNIX operating system. Various mobile phone operating systems, such as IOS and ANDROID, may also be utilized. It should be appreciated that other operating systems may also be utilized. The mass storage device 828 may store other system or application programs and data utilized by the computing device 800.

The mass storage device 828 or other computer-readable storage media may also be encoded with computer-executable instructions, which, when loaded into the computing device 800, transforms the computing device from a general-purpose computing system into a special-purpose computer capable of implementing the aspects described herein. These computer-executable instructions transform the computing device 800 by specifying how the CPU(s) 804 transition between states, as described above. The computing device 800 may have access to computer-readable storage media storing computer-executable instructions, which, when executed by the computing device 800, may perform the methods described herein related to one or more of imaging, machine learning, analyzing imaging, indicating diseases and/or conditions, or a combination thereof.

A computing device, such as the computing device 800 depicted in FIG. 8, may also include an input/output controller 832 for receiving and processing input from a number of input devices, such as a keyboard, a mouse, a touchpad, a touch screen, an electronic stylus, or other types of input device. Similarly, an input/output controller 832 may provide output to a display, such as a computer monitor, a flat-panel display, a digital projector, a printer, a plotter, or other types of output device. It will be appreciated that the computing device 800 may not include all of the components shown in FIG. 8, may include other components that are not explicitly shown in FIG. 8, or may utilize an architecture completely different than that shown in FIG. 8.

As described herein, a computing device may be a physical computing device, such as the computing device 800 of FIG. 8. A computing node may also include a virtual machine host process and one or more virtual machine instances. Computer-executable instructions may be executed by the physical hardware of a computing device indirectly through interpretation and/or execution of instructions stored and executed in the context of a virtual machine.

The computing device may be in communication with an imaging device 834. The imaging device may be any imaging device, such as an ultrasound imaging device. The imaging device 834 may scan a subject and generate imaging data based on the scan. The imaging data may be sent to the computing device 800. The computing device may process the imaging data by performing segmentation to determine one or more pleural line regions. The computing device may analyze the pleural line regions using a model, rules, a machine learning model, and/or the like. The pleural line regions may have various features recognized computed as values by the methods herein. The resulting values of features may be used to categorize the subject as having a condition, a disease, a disease level, or a combination thereof

The disclosure may include at least the following aspects.

Aspect 1. A method (e.g., a computer implemented method) comprising, consisting of, or consisting essentially of: receiving imaging data indicative of a lung of a subject; determining at least one pleural line region in the imaging data; determining one or more values (e.g., quantitative values) of one or more morphological features of the at least one pleural line region; and sending, based on the one or more values of one or more morphological features, an indication of a condition of the lung.

Aspect 2. The method of Aspect 1, wherein the indication of the condition comprises an indication of one or more of a disease, a level of a disease, severity of the disease, a viral disease, pneumonia, coronavirus disease, or a COVID-19 infection.

Aspect 3. The method of any one of Aspects 1-2, wherein the indication of the condition of the lung comprises one or more of an indication of one or more of the values of the one or more morphological features or an indication of a value determined based on one or more of the determined values of the one or more morphological features.

Aspect 4. The method of any one of Aspects 1-3, wherein the imaging data comprises lung ultrasound imaging data.

Aspect 5. The method of any one of Aspects 1-4, wherein the one or more morphological features comprise one or more of thickness, thickness variation, tortuosity, nonlinearity, or projected intensity variation.

Aspect 6. The method of any one of Aspects 1-5, wherein determining the one or more values of the one or more morphological features of the at least one pleural line region comprises performing feature extraction of a portion of the imaging data comprising the at least one pleural line region.

Aspect 7. The method of any one of Aspects 1-6, wherein sending the indication of the condition of the lung comprises one or more of sending the indication to a computing device, sending the indication to storage, or causing the indication of the condition to be output to via a display.

Aspect 8. The method of any one of Aspects 1-7, wherein determining the at least one pleural line region in the imaging data comprises performing automatic segmentation of the imaging data to detect the at least one pleural line region.

Aspect 9. The method of any one of Aspects 1-8, wherein determining the at least one pleural line region in the imaging data comprises receiving, based on user input, an indication of a location of the at least one pleural line region and segmenting, based on the indication of the location, the pleural line region.

Aspect 10. The method of any one of Aspects 1-9, wherein the pleural line region comprises a region of tissue having features within a threshold similarity to a line.

Aspect 11. The method of any one of Aspects 1-10, wherein determining the one or more values of the one or more morphological features comprises one or more of measuring or calculating of a value of a corresponding morphological feature based on intensity values of pixels of the imaging data comprising the pleural line region.

Aspect 12. The method of any one of Aspects 1-11, wherein determining the indication of the conditions comprises determining, based on applying one or more of a rule or a model to the one or more values of the morphological features, the indication of the condition.

Aspect 13. The method of any one of Aspects 1-12, wherein the one or more morphological features of the at least one pleural line region comprise features indicative of variations in one or more of shape or linearity of the at least one pleural line region.

Aspect 14. The method of any one of Aspects 1-13, further comprising training, based on a set of training images, a machine learning model configured to associate values of the one or more morphological features with corresponding indications of the condition, wherein the indication of the condition is determined based on the machine learning model.

Aspect 15. The method of any one of Aspects 1-14, further comprising determining, based on inputting the one or more values of the one or more morphological features to a machine learning model, the indication of the condition.

Aspect 16. The method of any one of Aspects 1-15, further comprising: applying weights to each of the one or more values of the one or more morphological features, wherein the weights are applied equally or based on a machine learning model; and averaging the weighted values to determine a value of the indication of the condition.

Aspect 17. A device comprising, consisting of, or consisting essentially of: one or more processors; and a memory storing instructions that, when executed by the one or more processors, cause the device to perform the methods of any one of Aspects 1-16.

Aspect 18. A non-transitory computer-readable medium storing instructions that, when executed by one or more processors, cause a device to perform the methods of any one of Aspects 1-16.

Aspect 19. A system comprising, consisting of, or consisting essentially of: an imaging device configured to determine imaging data indicative of a lung of a subject; and a computing device comprising one or more processors, and a memory, wherein the memory stores instructions that, when executed by the one or more processors, cause the computing device to perform the methods of any one of Aspects 1-16.

It is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Components are described that may be used to perform the described methods and systems. When combinations, subsets, interactions, groups, etc., of these components are described, it is understood that while specific references to each of the various individual and collective combinations and permutations of these may not be explicitly described, each is specifically contemplated and described herein, for all methods and systems. This applies to all aspects of this application including, but not limited to, operations in described methods. Thus, if there are a variety of additional operations that may be performed it is understood that each of these additional operations may be performed with any specific embodiment or combination of embodiments of the described methods.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described herein with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, may be implemented by computer program instructions. These computer program instructions may be loaded on a general-purpose computer, special-purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that may direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

The various features and processes described above may be used independently of one another, or may be combined in various ways. All possible combinations and sub-combinations are intended to fall within the scope of this disclosure. In addition, certain methods or process blocks may be omitted in some implementations. The methods and processes described herein are also not limited to any particular sequence, and the blocks or states relating thereto may be performed in other sequences that are appropriate. For example, described blocks or states may be performed in an order other than that specifically described, or multiple blocks or states may be combined in a single block or state. The example blocks or states may be performed in serial, in parallel, or in some other manner. Blocks or states may be added to or removed from the described example embodiments. The example systems and components described herein may be configured differently than described. For example, elements may be added to, removed from, or rearranged compared to the described example embodiments.

It will also be appreciated that various items are illustrated as being stored in memory or on storage while being used, and that these items or portions thereof may be transferred between memory and other storage devices for purposes of memory management and data integrity. Alternatively, in other embodiments, some or all of the software modules and/or systems may execute in memory on another device and communicate with the illustrated computing systems via inter-computer communication. Furthermore, in some embodiments, some or all of the systems and/or modules may be implemented or provided in other ways, such as at least partially in firmware and/or hardware, including, but not limited to, one or more application-specific integrated circuits (“ASICs”), standard integrated circuits, controllers (e.g., by executing appropriate instructions, and including microcontrollers and/or embedded controllers), field-programmable gate arrays (“FPGAs”), complex programmable logic devices (“CPLDs”), etc. Some or all of the modules, systems, and data structures may also be stored (e.g., as software instructions or structured data) on a computer-readable medium, such as a hard disk, a memory, a network, or a portable media article to be read by an appropriate device or via an appropriate connection. The systems, modules, and data structures may also be transmitted as generated data signals (e.g., as part of a carrier wave or other analog or digital propagated signal) on a variety of computer-readable transmission media, including wireless-based and wired/cable-based media, and may take a variety of forms (e.g., as part of a single or multiplexed analog signal, or as multiple discrete digital packets or frames). Such computer program products may also take other forms in other embodiments. Accordingly, the present invention may be practiced with other computer system configurations.

While the methods and systems have been described in connection with preferred embodiments and specific examples, it is not intended that the scope be limited to the particular embodiments set forth, as the embodiments herein are intended in all respects to be illustrative rather than restrictive.

It will be apparent to those skilled in the art that various modifications and variations may be made without departing from the scope or spirit of the present disclosure. Other embodiments will be apparent to those skilled in the art from consideration of the specification and practices described herein. It is intended that the specification and example figures be considered as exemplary only, with a true scope and spirit being indicated by the following claims.

### REFERENCES

1. WHO. Coronavirus disease (COVID-2019) situation reports. World Heal Organ. 2020.

2. Dong E, Du H, Gardner L. An interactive web-based dashboard to track COVID-19 in real time. Lancet Inf Dis. 2020;20(5):533-534.

3. Ai T, Yang Z, Hou H, et al. Correlation of chest CT and RT-PCR testing for coronavirus disease 2019 (COVID-19) in China: a report of 1014 cases. Radiology. 2020; 296(2):E32-E40.

4. Guan W-J, Ni Z-Y, Hu Y, et al. Clinical characteristics of coronavirus disease 2019 in China. N Engl J Med. 2020; 382(18):1708-1720.

5. Shi H, Han X, Jiang N, et al. Radiological findings from 81 patients with COVID-19 pneumonia in Wuhan, China: a descriptive study. Lancet Infect Dis. 2020; 20(4):425-434.

6. Zhang R, Ouyang H, Fu L, et al. CT features of SARS-CoV-2 pneumonia according to clinical presentation: a retrospective analysis of 120 consecutive patients from Wuhan city. Eur Radiol. 2020; 30(8):4417-4426.

7. Tung-Chen Y, Martí de Gracia M, Díez-Tascón A, et al. Correlation between chest computed tomography and lung ultrasonography in patients with coronavirus disease 2019 (COVID-19). Ultrasound Med Biol. 2020; 46(11):2918-2926.

8. Bouhemad B, Mongodi S, Via G, Rouquette I. Ultrasound for “lung monitoring” of ventilated patients. Anesthesiology. 2015.

9. Pesenti A, Musch G, Lichtenstein D, et al. Imaging in acute respiratory distress syndrome. Intensive Care Med United States. 2016; 42(5):686698.

10. Bouhemad B, Brisson H, Le-Guen M, Arbelot C, Lu Q, Rouby J-J. Bedside ultrasound assessment of positive end-expiratory pressure-induced lung recruitment. Am J Respir Crit Care Med United States. 2011; 183(3):341-347.

11. Bouhemad B, Liu Z-H, Arbelot C, et al. Ultrasound assessment of antibiotic-induced pulmonary reaeration in ventilator-associated pneumonia. Crit Care Med United States. 2010; 38(1):84-92.

12. Soummer A, Perbet S, Brisson H, et al. Ultrasound assessment of lung aeration loss during a successful weaning trial predicts postextubation distress*. Crit Care Med United States. 2012; 40(7):20642072.

13. Sultan L R, Sehgal C M. A review of early experience in lung ultrasound in the diagnosis and management of COVID-19. Ultrasound in Medicine & Biology. 2020; 46(9):2530-2545.

14. Huang Y, Wang S, Liu Y, et al. A preliminary study on the ultrasonic manifestations of peripulmonary lesions of non-critical novel coronavirus pneumonia (COVID-19). SSRN Electronic Journal. https://doi.org/10.2139/ssrn.3544750.

15. Hui D S, I Azhar E, Madani T A, et al. The continuing 2019-nCoV epidemic threat of novel coronaviruses to global health—The latest 2019 novel coronavirus outbreak in Wuhan. China Int J Infect Dis. 2020; 91:264-266.

16. Inchingolo R, Smargiassi A, Moro F, et al. The diagnosis of pneumonia in a pregnant woman with COVID-19 using maternal lung ultrasound. Am J Obstet Gynecol. 2020.

17. Kalafat E, Yaprak E, Cinar G, et al. Lung ultrasound and computed tomographic findings in pregnant woman with COVID-19. Ultrasound Obstet Gynecol. 2020; 55(6):835-837.

18. Tung-Chen Y. Lung ultrasound in the monitoring of COVID-19 infection. Clin Med. 2020; 20(4): e62-e65.

19. Pinto A, Pinto F, Faggian A, et al. Sources of error in emergency ultrasonography. Crit Ultrasound J. 2013; 5(Suppl 1):S1.

20. Moshavegh R, Hansen K L, Moller-Sorensen H, Nielsen M B, Jensen J A. Automatic detection of B-Lines in in vivo lung ultrasound. IEEE Trans Ultrason Ferroelectr Freq Control. 2019; 66(2):309-317.

21. Van Sloun R J G, Demi L. Localizing B-lines in lung ultrasonography by weakly supervised deep learning, in-vivo results. IEEE J Biomed Heal Informatics. 2020; 24(4):957-964.

22. Correa M, Zimic M, Barrientos F, et al. Automatic classification of pediatric pneumonia based on lung ultrasound pattern recognition. PLoS One Public Library of Science. 2018; 13(12):1-13.

23. Millington S J, Koenig S, Mayo P, Volpicelli G. Lung ultrasound for patients with coronavirus disease 2019 pulmonary disease. Chest. 2021; 159(1):205-211.

24. Tremeau A, Borel N. A region growing and merging algorithm to color segmentation. Pattern Recognit. 1997; 30(7):1191-1203.

25. Kairuddin WNHW, Mahmud WMHW. Texture feature analysis for different resolution level of kidney ultrasound images. IOP Conf Ser Mater Sci Eng. 2017; 226:12136.

26. Johnson W D, Koch G G. Intraclass Correlation Coefficient. In: Lovric M. (eds) International Encyclopedia of Statistical Science. Berlin, Heidelberg: Springer, 2011. https://doi.org/10.1007/978-3-642-04898-2.

27. Cicchetti D V. Guidelines, criteria, and rules of thumb for evaluating normed and standardized assessment instruments in psychology. Psychol Assess. 1994; 6(4):284-290.

28. Trauer M M, Matthies A, Mani N, et al. The utility of lung ultrasound in COVID-19: a systematic scoping review. Ultrasound. 2020; 28(4):208-222. https://doi.org/10.1177/1742271x20950779.

29. Hankins A, Bang H, Walsh P. Point of care lung ultrasound is useful when screening for CoVid-19 in emergency department patients. medRxiv Prepr Sery Heal Sci. 2020.

30. Mongodi S, Bouhemad B, Orlando A, et al. Modified lung ultrasound score for assessing and monitoring pulmonary aeration Modifizierter Lungen-US-Score zur Bewertung and Überwachung der Belüftung der Lunge. Modif Lung Ultrasound Ultraschall Med. 2017.

31. Onigbinde S O, Ojo A S, Fleary L, Hage R. Chest Computed tomography findings in COVID-19 and influenza: a narrative review. Biomed Res Int. 2020; 2020.6928368. https://europepmc.org/articles/PMC7275219.

32. TMC deL, A F daSS, BR deL, ME deAB, J deAS. Mechanism of inflammatory response in associated comorbidities in COVID-19. Diabetes Metab Syndr Clin Res Rev. 2020.

33. Alfuraih A M. Point of care lung ultrasound in COVID-19: hype or hope? BJR Open. 2020; 2(1):20200027.

What is claimed:

1. A method, comprising:
receiving imaging data indicative of a lung of a subject;
determining at least one pleural line region in the imaging data;
determining one or more values of one or more morphological features of the at least one pleural line region; and
sending, based on the one or more values of one or more morphological features, an indication of a condition of the lung.

2. The method of claim 1, wherein the indication of the condition comprises an indication of one or more of a disease, a level of a disease, a severity of the disease, a viral disease, pneumonia, coronavirus disease, or a COVID-19 infection.

3. The method of claim 1, wherein the indication of the condition of the lung comprises one or more of an indication of one or more of the values of the one or more morphological features or an indication of a value determined based on one or more of the determined values of the one or more morphological features.

4. The method of claim 1, wherein the imaging data comprises lung ultrasound imaging data.

5. The method of claim 1, wherein the one or more morphological features comprise one or more of thickness, thickness variation, tortuosity, nonlinearity, or projected intensity variation.

6. The method of claim 1, wherein determining the one or more values of the one or more morphological features of the at least one pleural line region comprises performing feature extraction of a portion of the imaging data comprising the at least one pleural line region.

7. The method of claim 1, wherein sending the indication of the condition of the lung comprises one or more of sending the indication to a computing device, sending the indication to storage, or causing the indication of the condition to be output to via a display.

8. The method of claim 1, wherein determining the at least one pleural line region in the imaging data comprises performing automatic segmentation of the imaging data to detect the at least one pleural line region.

9. The method of claim 1, wherein determining the at least one pleural line region in the imaging data comprises receiving, based on user input, an indication of a location of the at least one pleural line region and segmenting, based on the indication of the location, the pleural line region.

10. The method of claim 1, wherein the pleural line region comprises a region of tissue having features within a threshold similarity to a line.

11. The method of claim 1, wherein determining the one or more values of the one or more morphological features comprises one or more of measuring or calculating of a value of a corresponding morphological feature based on intensity values of pixels of the imaging data comprising the pleural line region.

12. The method of claim 1, wherein determining the indication of the conditions comprises determining, based on applying one or more of a rule or a model to the one or more values of the morphological features, the indication of the condition.

13. The method of claim 1, wherein the one or more morphological features of the at least one pleural line region comprise features indicative of variations in one or more of shape or linearity of the at least one pleural line region.

14. The method of claim 1, further comprising training, based on a set of training images, a machine learning model configured to associate values of the one or more morphological features with corresponding indications of the condition, wherein the indication of the condition is determined based on the machine learning model.

15. The method of claim 1, further comprising determining, based on inputting the one or more values of the one or more morphological features to a machine learning model, the indication of the condition.

16. The method of claim 1, further comprising:
applying weights to each of the one or more values of the one or more morphological features, wherein the weights are applied equally or based on a machine learning model; and
averaging the weighted values to determine a value of the indication of the condition.

17. A device comprising:
one or more processors; and
a memory storing instructions that, when executed by the one or more processors, cause the device to:
receive imaging data indicative of a lung of a subject;
determine at least one pleural line region in the imaging data;
determine one or more values of one or more morphological features of the at least one pleural line region; and
send, based on the one or more values of one or more morphological features, an indication of a condition of the lung.

18. The device of claim 17, wherein the one or more morphological features comprise one or more of thickness, thickness variation, tortuosity, nonlinearity, or projected intensity variation.

19. A system comprising:
an imaging device configured to determine imaging data indicative of a lung of a subject; and
a computing device configured to:
receive the imaging data indicative of the lung of the subject;
determine at least one pleural line region in the imaging data;
determine one or more values of one or more morphological features of the at least one pleural line region; and
send, based on the one or more values of one or more morphological features, an indication of a condition of the lung.

20. The system of claim 19, wherein the one or more morphological features comprise one or more of thickness, thickness variation, tortuosity, nonlinearity, or projected intensity variation.
